(19) United States
(12) Patent Application Publication
(10) Pub. No.: US 2025/0109077 A1
(43) Pub. Date: Apr. 3, 2025

(54) YEAST-BASED FERTILIZER

(71) Applicant: Chemical Evolution Ltd., Calgary (CA)

(72) Inventors: Corynne O'FARRELL, Calgary (CA); Alejandra ENRIQUEZ, Calgary (CA); Anastasia SOSKOVETS, Calgary (CA); Markus WEISSENBERGER, Calgary (CA)

(21) Appl. No.: 18/900,070

(22) Filed: Sep. 27, 2024

(30) Foreign Application Priority Data
Sep. 29, 2023 (CA) 3214892

(51) Int. Cl. C05F 11/08; C05F 17/20; C12N 1/18

(52) U.S. Cl. CPC C05F 11/08; C05F 17/20; C12N 1/18

(57) ABSTRACT

A process to make a yeast-based fertilizer composition from a combination of Brewer's Spent Yeast and a second organism, said process comprising the steps of:
providing said live Brewer's Spent yeast material;
exposing said live Brewer's Spent yeast material to said second organism, wherein said second organism is capable of degrading proteins and performing aerobic respiration, thereby creating a live mixed culture;
incubating said live mixed culture in the presence of at least one nitrogen-containing compound thereby creating an incubation mixture;
optionally incorporating into said incubation mixture at least one additional nutrient to promote the propagation of said live mixed culture;

optionally, injecting air into said incubation mixture so as to minimize and/or substantially inhibit the production of ethanol;

wherein said incubation mixture undergoes incubation for a period of time sufficient for said live mixed culture to metabolize said at least one nitrogen-containing compound and for said live mixed culture to propagate and resulting in a pre-lysis mixture comprising metabolized nitrogen-containing compounds and said live mixed culture;
autolyzing said pre-lysis mixture under specific conditions yielding an autolyzed mixture which comprises said yeast-based fertilizer composition.

### FIELD OF THE INVENTION

The present invention is directed to a yeast-based fertilizing product having an enhanced nitrogen content wherein the method of preparation involves spiking a first yeast population with at least one other organism.

### BACKGROUND OF THE INVENTION

Each year, agro-industrial activities produce large quantities of agro-industrial organic by-products resulting from activities as diverse as the wine industry, beer, meat production, flour, rice, dairy, etc. These activities generate large quantities of by-products, which is problematic due to their lack of use as well as accumulation in the environment. Many of these by-products end up in municipal landfills or wastewater treatment plants, where they create serious environmental and equipment problems due to microbial decomposition, contamination, and leachate production. From an economic standpoint, there is an additional cost related to the handling of waste and its incineration or disposal, leading to large amounts of greenhouse gas emissions. There is constant and growing pressure from political groups, as well as environmental entities, to take steps to reduce pollution.

In spite of their diverse origin, agro-industrial by-products are characterized by high organic matter content, including high amounts of protein. One of the main areas of use of waste protein material is in the production of nutritional products for plants. Waste protein materials are used as a nitrogen source for soil in the form of fertilizers. Fertilizers are organic or inorganic substances, of natural or synthetic origin, that are used to enrich soil and provide plants with one or more nutritional elements essential for plant development.

Insufficient supply of nitrogen to crops results in smaller leaves, as well as reduction in chlorophyll production and chloroplast development. This can potentially lead to chlorosis of the entire crop. Plant growth can also be stunted because of the lack of nitrogen.

Organic fertilizers are not only a great source of nutrients for plants, but they also provide nutrients for the microorganisms in the soil, thereby improving soil health. In instances of poor soil quality, organic fertilizers contribute to the physical, chemical, and biological properties of the soil. In the food and beverage industry, yeast is used for: baking, alcohol production, feed protein, health food raw materials, single-cell protein, vitamins, and nucleic acid-related substances. Recently, there has been a renewed interest in using yeast as a fertilizing agent in the agro-industrial sector.

One of the abundant sources for organic matter is Brewer's Spent Yeast (BSY) (also known as residual yeast or surplus yeast). This refers to a prevalent by-product of the brewing industry, created when the yeast used in fermentation is no longer useful, and must be disposed of. BSY is mainly composed of the yeast strain Saccharomyces cerevisiae, although hundreds of strains are known to be involved in the alcohol fermentation process. Yeast cells contain a wide range of different functional components including peptides, amino acids, polyphenols, carotenoids and flavonoids. These components impart bioactive properties to the yeast extract once extracted from the cell. Yeast degradation yields a wide variety of compounds, vitamins, and minerals. It may yield upwards of: 40% protein, less than 1% in fat, close to 40% of carbohydrates, and various other components rounding out the remaining portion. Among the protein portion, amino acids have been analyzed to account for varying amounts (based on the yeast strain) ranging from close to 0.4% to upwards of 7%. These amino acids include: Lysine (Lys); Methionine (Met); Tryptophan (Trp); Arginine (Arg); Histidine (His); Isoleucine (Ile); Leucine (Leu); Phenylalanine (Phe); Threonine (Thr); Valine (Val); Glycine (Gly); Cysteine (Cys); Tyrosine (Tyr); Alanine (Ala); Serine (Ser); Aspartic Acid (Asp); and Glutamic Acid (Glu).

Preferably, the discarded BSY material consists of yeasts and by-products from the alcoholic fermentation of barley malt. It consists of minerals, traces of fatty acids, carbohydrates, and other compounds. Some of the minerals present include: phosphorus, potassium, sulfur, magnesium, calcium or sodium. Traces of lipids include: lecithins, cephalins, and other saturated and unsaturated fatty acids. Carbohydrates such as: glycogen, trehalose, glucans or mannans, and other compounds, such as: ethyl alcohol, carbon dioxide, traces of esters, aldehydes, ketones and higher alcohols, etc. These residues may be suspended in beer as they leave the industrial plant, without the need for drying or concentration, or be in solid form once the remaining beer has been removed. The remaining beer can be separated by any conventional method such as filtration, sedimentation or centrifugation.

Certain jurisdictions with a low number of fermentation facilities would require an additional source of yeast for an increased production of a yeast-based fertilizing product. That is why it is imperative to find alternatives that are compatible to the discarded BSY and that will allow for fertilizer production.

Many patent applications and patents discuss various uses of yeasts for such applications. A few of them are set out below to provide an overview of the field. Hungarian patent document HU 9902060 discloses the composition of an aqueous fertilizer for the leaves and roots of plants containing yeast of the genus Saccharomyces, trace elements, complexing agents, buffering agents and other nutrients such as amino acids, humic acids, enzymes, carbohydrate sources, etc.

Chinese patent application CN19191800 describes a nutrient for animals and plants that is prepared by diluting Saccharomyces cerevisiae sludge in water until an emulsion is obtained; mixing this with papain, neutral proteases and sodium chloride; hydrolyzing the mixture afterwards; inactivating enzymes; and finally, concentrating or drying the product obtained. The final product contains a large amount of nutrients.

Chinese patent application CN 191 1870 describes a plant nutrient that improves soil microorganisms, promotes plant growth, increases fertilizer utilization rates, and increases plant resistance to disease and stress. This nutrient is composed of Saccharomyces cerevisiae, Lactobacillus plantarum, Lactobacillus acidophilus, and other components such as potato or coffee derivatives, glucose, peptone, magnesium and manganese sulfates, dipotassium hydrogen phosphate, and sodium chloride.

US patent application 2003/022357 discloses a biological fertilizer containing magnetically activated yeast cells of the genus Saccharomyces and sludge from wastewater treatment or storage.

US patent application no. 2002/187900 discloses a biological fertilizer comprised of electromagnetically activated yeast cells of the genus Saccharomyces and cattle manure.

US patent application no. 2009/0173122A1 discloses a soluble, liquid or dry fertilizer for application to a plant or soil that is grown or farmed as “organic” as defined under the USDA National Organic Program Rule. The fertilizer is produced from distiller's yeast from beer and/or alcohol production. The yeast cells are autolyzed using heat and the autolysates are separated by centrifugation into insoluble cell walls and cellular plasma. The plasma is concentrated by evaporation into the fertilizer. It also stated that the fertilizer may be further processed by proteolytic enzyme (protease) lysis to produce smaller-sized, soluble, nitrogen-containing compounds including protein, peptides, amino acids, amines and ammonia. The fertilizer has a solids content between ten and sixty-five percent, a total protein content of at least ten percent and up to eighty-five percent, a total Nitrogen content between one and fourteen percent, and a pH between 2.5 and 10.

In light of the state of the art, there exists a need to improve the production of yeast-based organic fertilizers using, in some instances, mixed yeast-based starting materials. This is true especially in jurisdictions where access to fertilizers or access to brewing facilities is limited.

### SUMMARY OF THE INVENTION

According to an aspect of the present invention, there is provided a process for obtaining organic extracts from residues of the beer industry and combining them with another organism or organisms in a way that the processed material can be used as bio-stimulants and biofertilizers in agriculture, preferably organic farming.

According to a first aspect of the present invention, there is provided a process to make a yeast-based fertilizer composition from a combination of Brewer's Spent Yeast and a second organism, said process comprising the steps of:
providing said live Brewer's Spent yeast material;
exposing said live Brewer's Spent yeast material to said second organism, wherein said second organism is capable of degrading proteins and performing aerobic respiration, thereby creating a live mixed culture;
incubating said live mixed culture in the presence of at least one nitrogen-containing compound thereby creating an incubation mixture;
optionally incorporating into said incubation mixture at least one additional nutrient to promote the propagation of said live mixed culture;
optionally, injecting air into said incubation mixture so as to minimize and/or substantially inhibit the production of ethanol;
   wherein said incubation mixture undergoes incubation for a period of time sufficient for said live mixed culture to metabolize said at least one nitrogen-containing compound and for said live mixed culture to propagate and resulting in a pre-lysis mixture comprising metabolized nitrogen-containing compounds and said live mixed culture;
autolyzing said pre-lysis mixture under specific conditions yielding an autolyzed mixture which comprises said yeast-based fertilizer composition.

According to a preferred embodiment of the present invention, the process further comprises a step of dehydrating or evaporating said autolyzed mixture to yield said yeast-based fertilizer composition with a pre-determined NPK number. The NPK number reflects the proportion of three plant nutrients, nitrogen (N), phosphorus (P), and potassium (K), by percentage by weight.

According to a preferred embodiment of the present invention, said yeast-based fertilizer composition is filtered to yield a liquid free of solids having a particle size of 75 microns or greater.

Preferably, said secondary organism capable of degrading proteins and performing aerobic respiration is a fungus. More preferably, said fungus is an organism belonging to the genus Saccharomyces.

According to a preferred embodiment of the present invention, said at least one nitrogen-containing compound is a naturally occurring organic protein source. Preferably, said at least one nitrogen-containing compound is selected from the group consisting of: proteins; amino acids; peptides; salts thereof and combinations thereof. More preferably, said at least one nitrogen-containing compound is selected from the group consisting of: gelatin; tryptone; peptone; collagen; casein; yeast extract; bloodmeal; bonemeal; soybean meal; Lysine (Lys); Methionine (Met); Tryptophan (Trp); Arginine (Arg); Histidine (His); Isoleucine (Ile); Leucine (Leu); Phenylalanine (Phe); Threonine (Thr); Valine (Val); Glycine (Gly); Cysteine (Cys); Tyrosine (Tyr); Alanine (Ala); Glutamine (Gln); Glycine (Gly); Serine (Ser); Asparagine (Asn); Aspartic Acid (Asp); and Glutamic Acid (Glu); and a combination thereof and/or salts thereof.

According to a preferred embodiment of the present invention, said at least one additional nutrient for the propagation of the live mixed culture is selected from the group consisting of: carbohydrates; vitamins; organic salts; inorganic salts; trace metals and a combination thereof. Preferably, at least one additional nutrient for the propagation of the live mixed culture is a carbohydrate.

According to a preferred embodiment of the present invention, the step of incubating has a duration ranging from 3 to 48 hours. According to a more preferred embodiment of the present invention, the step of incubating has a duration ranging from 6 to 24 hours.

According to a preferred embodiment of the present invention, the step of incubating is carried out at a temperature ranging from 10 to 40° C. More preferably, the step of incubating is carried out at a temperature ranging from 20 to 30° C. Even more preferably, the step of incubating is carried out at a temperature ranging from 25 to 30° C.

According to a preferred embodiment of the present invention, the step of autolyzing (or lysis) is carried out at a temperature ranging from 40 to 100° C. Preferably, the step of autolyzing is carried out at a temperature ranging from 45 to 60° C. It is known by those skilled in the art that should the temperature during lysis be above a certain temperature it would occur too quickly, while if the temperature during the lysis be below another certain temperature the lysis would occur too slowly and the organisms consume the nutrients which would, in turn, lower the expected yields.

According to a preferred embodiment of the present invention, a quantity of said nitrogen source added to said live mixed culture in solution ranges from 0.5 to 20% of the total fertilizer composition. Preferably, the nitrogen source added to the live mixed culture in solution ranges from 0.5 and 15 wt. % of the total fertilizer composition. Even more preferably, the nitrogen source added to the live mixed culture in solution ranges from 0.5 and 10 wt. % of the total fertilizer composition.

According to a preferred embodiment of the present invention, the live mixed culture has a dry content ranging from 0 and 30 wt. % of the total fertilizer composition. Preferably, live mixed culture has a dry content ranging from 7 and 20 wt. % of the total fertilizer composition.

According to a preferred embodiment of the present invention, the process further comprises a step of incorporating at least one low molecular weight nitrogen-containing compound to said pre-lysis mixture prior to the step of autolyzing. Preferably, said at least one low molecular weight nitrogen-containing compound is selected from the group consisting of: oligopeptides, amino acids, and combinations thereof and/or salts thereof.

According to another aspect of the present invention, there is provided a yeast-based fertilizer composition derived from an incubation of a first organism with a second organism, wherein said first and second organisms are capable of degrading proteins and performing aerobic respiration, and wherein a Brewer's Spent yeast material comprises said first organism and said second organism comes from a different source. Preferably, the incubation of at least said first and said second organisms capable of degrading proteins and perform aerobic respiration, is preceded by an addition of at least one nitrogen-containing compound. Preferably, said second organism is selected from the genus Saccharomyces.

According to a preferred embodiment of the present invention, the incubation of at least two organisms capable of degrading proteins and perform aerobic respiration is preceded by an addition of at least one additional nutrient for the propagation of the live culture selected from the group consisting of: carbohydrates; vitamins; organic salts; inorganic salts; trace metals and a combination thereof. Preferably, said at least one nitrogen-containing compound is selected from the group consisting of: proteins; amino acids; peptides; amine salts thereof and a combination thereof. More preferably, said at least one nitrogen- containing compound is selected from the group consisting of: gelatin; tryptone; peptone; collagen; casein; yeast extract; bloodmeal; bonemeal; soybean meal; Lysine (Lys); Methionine (Met); Tryptophan (Trp); Arginine (Arg); Histidine (His); Isoleucine (Ile); Leucine (Leu); Phenylalanine (Phe); Threonine (Thr); Valine (Val); Glycine (Gly); Cysteine (Cys); Tyrosine (Tyr); Alanine (Ala); Glutamine (Gln); Glycine (Gly); Serine (Ser); Asparagine (Asn); Aspartic Acid (Asp); and Glutamic Acid (Glu); and a combination thereof and/or salts thereof.

According to a preferred embodiment of the present invention, a nitrogen content ranging between 0.5 and 10 wt. %.

### Phosphorous Content

According to a preferred embodiment of the present invention, there is provided a fertilizer wherein the phosphorous content is adjusted to meet a certain demand by altering the quantity of BSY and other organisms in the initial process. It is known to the person skilled in the art that there are situations wherein a fertilizer with little to no phosphorous is desired. For example, if the soil is already rich in phosphorous, adding more could disrupt the balance of nutrients and lead to reduction of micronutrient uptake resulting in poor plant health. Additionally, there are some species of plants that have a low phosphorous requirement and may suffer from phosphorous toxicity if the concentration of phosphorous provided is elevated. High phosphorous content in soil has also been shown to lead to inhibition of early seedling growth and may need to be avoided during the early growth stage. In these cases, when a low phosphorous fertilizer is desired, the amount of phosphorous in the final product can be adjusted by altering the concentrations of BSY and the at least one organism in the manufacturing.

### Particle Size

Currently, Brewer's Spent Yeast may be used as a fertilizer by spreading it directly in the field. However, this method does not maximize on the potential of the nitrogen in the fertilizer as it is not processed (read metabolized) to become more bioavailable. The consequences of this include accumulation in the soil and potential acidification or alkalinization depending on the content of the BSY and its metabolism by soil organisms and plants. As such, post-processing steps, although optional, are important for the application of the fertilizer to the soil or plant and its uptake. According to a preferred embodiment of the present invention, the nitrogen present in BSY is made more bioavailable by the process described herein. The nitrogen extracted into soluble, smaller molecules is subsequently more easily absorbed by plants for their growth.

Traditionally, BSY can be spread directly on the field as a fertilizer (as a slurry), however, the application process is time consuming and requires manpower. The process described in this invention not only maximizes on the nitrogen source, but also produces a liquid fertilizer that is optionally filtered through a 75 μm filter. This additional filtration step allows the fertilizer composition to be used in most drip/spray irrigation systems which are commonly used both in fields and in greenhouses, therefore reducing the manpower necessary for fertilization and increasing crop yield and financial profits.

Preferably, the process further comprises a step of exposing yeast to added nutrients prior to lysis of said yeast.

After undergoing autolysis, yeast extracts contain practically all the hydrolyzed protein in the form of free amino acids, oligopeptides and other peptides of greater molecular weight and, in addition, practically all the nutrients of the starting residue. These extracts are highly prized and have a great potential as biofertilizers and bio-stimulants. Moreover, they are highly sought after as they fulfill the requirements to be used in organic farming settings.

### DETAILED DESCRIPTION OF AN EMBODIMENT OF THE PRESENT INVENTION

The description that follows, and the embodiments described therein, is provided by way of illustration of an example, or examples, of particular embodiments of the principles of the present invention. These examples are provided for the purposes of explanation, and not limitation, of those principles and of the invention.

Nitrogen is a basic constituent of proteins, nucleic acids, cell components, etc. and, therefore, encourages the development of the metabolic activity of plants and microorganisms. Amino acids, oligopeptides and peptides of low molecular weight constitute nutritious substances, which can be taken up and assimilated easily by plants. Fertilizers containing these compounds can be applied on the leaves of plants or to the root system where they will be transported to the flowers, fruits, etc. to provide essential nutrients for the development of the plant. According to a preferred embodiment of the present invention, there is provided a process to make a yeast-based fertilizer, which is to be understood, as being a fertilizer manufactured through the use of a combination of two or more yeast cultures.

Brewer's Spent Yeast (BSY) is a by-product of the brewing industry. According to a preferred embodiment of the present invention, a quantity of nitrogen supplement that can be a protein or a mixture of amino acids is fed to a live culture comprising the BSY and at least one other organism capable of degrading proteins and perform aerobic respiration, thereby creating a live mixed culture. The incubation mixture is left for a period of approximately 3-48 hours, at a certain temperature, to incubate under aerobic conditions. During that period, the live culture degrades the proteins and polypeptides into its constituent amino acids, which makes the nitrogen readily bioavailable for plants and the like. At the end of the 3-48 hours, the live mixed culture is forced into autolysis. After lysis, if required, the lysed mixture is administered an additional mixture of amino acids to further increase the nitrogen content in the liquid solution of lysed mixture. In some embodiments of the present invention, post-processing steps are added post-lysis to meet specifications. Examples of those post-processing steps include filtration, centrifugation, evaporation, sterilization, etc.

The pH of the thus obtained fertilizing solution is between 4.0 and 7.0. The nitrogen content is about 1-10% wt. These amounts can vary depending on the quantity of nitrogen sources added during the first and second addition. Moreover, the type of nitrogen source used during the addition will also have a direct impact on the ultimate nitrogen content of the liquid fertilizer.

According to a preferred embodiment of the present invention, there is provided a process to make a yeast-based fertilizer manufacturing through the use of two or more yeast cultures. According to a preferred embodiment of the present invention, there is provided a yeast-based fertilizer which is organic or can be labelled as organic. Preferably, the yeast-based fertilizer is in liquid form, or can be diluted and dispensed on or near plants, or foliage, or roots. Preferably, applying such a composition will allow nitrogen to be readily available for uptake by plants.

According to a preferred embodiment of the present invention, the compounds that may be used to increase the nitrogen content of the yeast include amino acids, peptides, proteins, heterocycles, substituted amines, other industrial by-products, and yeast extracts. Preferably, the compounds that are amino acids to be selected from the group consisting of: alanine; arginine; asparagine; aspartic acid; cysteine; glutamic acid; glutamine; glycine; histidine; isoleucine; leucine; lysine; methionine; phenylalanine; proline; serine; threonine; tryptophan; tyrosine; and valine.

According to a preferred embodiment of the present invention, the amino acids can be added as is, or as a salt thereof. Preferably, the amino acids used have a high N:C ratio. Amino acids including but not limited to: glycine, cysteine, serine, and alanine (1 nitrogen atom); lysine, asparagine, tryptophan, and glutamine (each have 2 nitrogen atoms); arginine (4 nitrogen atoms); histidine (3 nitrogen atoms) are particularly preferred. Most preferred, is lysine sulfate. Lysine sulfate is commonly produced by bacterial fermentation and is typically used as a feed additive to farm animals, poultry, and fish. As such, it is considerably cheaper than other sources of lysine and moreover, it readily dissolves in water.

According to a preferred embodiment of the present invention, the resulting fertilizer may be dried into a soluble solid. According to a preferred embodiment of the present invention, the liquid fertilizer solution may be spray dried into a powder, oven dried, or granulated. According to a preferred embodiment of the present invention, the liquid fertilizer can also be concentrated by evaporation or diluted to a more soluble state.

It is noteworthy to mention that, according to a preferred embodiment of the present invention, the conversion of a carbohydrate source into ethanol is avoided as much as possible, as the target products are high nitrogen-containing compounds. In aerobic respiration, the yeast converts carbon sources to CO₂. Removal of carbon and oxygen through this approach, in theory, results in an increase in the concentration of nitrogen in the system.

According to a preferred embodiment of the present invention, the resulting mixture is concentrated. Preferably, the removal of the insoluble solids may be done using filters, decanters or centrifuges. Preferably, concentration may be achieved by using equipment such as evaporators, ovens, or membrane filters.

According to a preferred embodiment of the present invention, the resulting yeast-based fertilizer product has the following characteristics: solids content between 10 and 80% on a weight-to-weight basis; a total nitrogen content between 0.5 and 10% on a weight-to-weight basis; and a final pH of between 4.0 and 7.0.

Preferably, the fertilizer is stable at normal environmental temperatures and requires no special handling.

According to a preferred embodiment, the yeast vacuolar proteases Cerevisin (EC 3.4.21.48, yeast proteinase B, proteinase yscB, baker's yeast proteinase B, brewer's yeast proteinase, peptidase beta), which are active at two different pH ranges, are used to break down the proteins obtained during autolysis of yeast cells from higher molecular weight proteins to lower molecular weight peptides. Thus, avoiding an external addition of proteases like papain to denature the complex proteins. Cerevisin, which is a yeast internal protease, is used to break down the externally added protein like gelatin, pepsin etc., and make the nitrogen more available for plants.

The following examples illustrate the invention and should not be considered as limiting its scope.

For the purpose of these experiments, gelatin was used as a model protein to demonstrate the protein-degrading ability of the selected microorganisms. Gelatin was used as it is an abundant, cheap nitrogen source that is a co-product of the meat processing industry and widely available in many jurisdictions worldwide. In this example, sucrose was employed as the additional nutrient to promote the growth of the culture as an inexpensive carbohydrate source. The use of gelatin and sucrose within this invention is exemplary only and in no way is it meant to limit the nitrogen-containing proteins as well as addition nutrients to promote culture growth that can be used for the purpose of this invention, nor to limit the scope of the invention.

### EXAMPLES

### Experiment #1

Experiment 1 demonstrates that Baker's yeast as an alternative source of yeast can be employed to digest a nitrogen-containing compound and increase nitrogen concentration in the fertilizer. Increasing amounts of commercially available Baker's yeast were combined with 700 mL of water, 10 g of sucrose and 20 g of gelatin (see Table 1). All experiments were done in duplicate. All samples were incubated for 24 hours at 20-30° C. with a subsequent lysis step at 50° C. for 24 hours. Cell count was performed on all samples after incubation (see Table 1). After lysis the samples were cooled down to room temperature and were evaluated visually, then tested for nitrogen concentration testing.

To account for nitrogen in the starting yeast, all values of experimental nitrogen concentration have been normalized by subtracting the nitrogen contributed from the yeast. It will be known to those skilled in the art that some degree of evaporation occurs during incubation and lysis which will decrease the overall mass of the final sample. In some preferred embodiments, the concentrated liquid fertilizer contains 60% water and 40% solids.

TABLE 1
Sample composition with cell counts after incubation and final nitrogen
concentrations. Normalized nitrogen concentration calculated by
subtracting the nitrogen contributed from the dry yeast.

| | | Samples | | Normalized |
|---|---|---|---|---|
| | Baker's Yeast | diluted with | Live Cell count | Nitrogen (%) |
| Sample ID | (g) | water (mL) | (cells/mL) | in sample |
| S1 | 1 | 700 | 1.42E+07 | 0.52 |
| S2 | 10 | 700 | 2.01E+08 | 0.52 |
| S3 | 50 | 700 | 5.00E+08 | 0.61 |
| S4 | 100 | 700 | 1.65E+09 | 0.89 |
| S5* | 250 | 1000 | 3.78E+09 | 0.69 |

*Samples S5 used 1000 mL of water due to poor solubility of yeast.

Table 2 shows the consistency of the different samples after lysis. It is known for those skilled in the art, that gelatin would solidify, if not broken down. For the purpose of this invention, a liquid fertilizer is desired; thus, liquid consistency is a desirable specification for manufacturing. As a rule of thumb an acceptable liquid, will, upon visual observation, exhibit no observable viscosity.

TABLE 2
Sample consistency after lysis. Samples were
consistent between replicates.

| | | Estimation of Degradation |
|---|---|---|
| Sample ID | Consistency after lysis | as compared to controls |
| S1 | Jelly-like consistency, samples | <5% |
| | mostly solid | |
| S2 | Jelly-like consistency | 25% |
| S3 | Highly viscous liquid | 50% |
| S4 | Viscous liquid | 90% |
| S5 | Liquid | >95% |

A higher concentration of Baker's yeast resulted in more gelatin degradation observed (Table 2). This suggests that the activity and number of alive yeast cells in the solution play a crucial role in the breakdown of gelatin, to a point where the addition of more live yeast cells cause a decrease in nitrogen, likely due to increase cell density and lack of nutrients. This experiment demonstrates that Baker's yeast successfully degrades gelatin to a point where it will not solidify. The nitrogen content of samples S1 to S5 was analyzed and quantified. Table 3 provides the nitrogen content obtained from samples S1 to S5 after normalization.

TABLE 3
Normalized nitrogen content from gelatin
degradation experiment

| Sample ID | Normalized N (wt. %) |
|---|---|
| S1 | 0.52 |
| S2 | 0.52 |
| S3 | 0.61 |
| S4 | 0.89 |
| S5 | 0.69 |

### Experiment #2

Two different amounts of Baker's yeast were tested and combined with 700 ml of water, 10 g of sucrose and 20 g of gelatin (see Table 4). Samples were incubated for two different times (Table 4). After incubation, all samples were lysed at 50° C. for 24 hours. After lysis the samples were cooled down to room temperature and tested for nitrogen concentration. It will be known to those skilled in the art, that some degree of evaporation occurs during incubation and lysis decreasing the overall mass of the final sample.

TABLE 4
Composition of samples and their corresponding incubation
times, final consistency, and nitrogen concentration.

| | Baker's | | | Theoretical | Nitrogen |
|---|---|---|---|---|---|
| | Yeast | Incubation | Consistency | N (%) in | (%) in |
| Sample ID | (g) | time (h) | of sample | sample | sample |
| S6a | 50 | 0 | Jelly-like | 0.9 | 1.05 |
| S6b | 50 | 3 | Viscous liquid | 0.9 | 1.58 |
| S7a | 100 | 0 | Jelly-like | 1.2 | 1.54 |
| S7b | 100 | 3 | Viscous liquid | 1.2 | 2.02 |

The data set out in Table 4 indicates that the incubation step is crucial for the degradation of gelatin and the concentration of nitrogen in the final product. This is demonstrated by the final nitrogen concentration of the samples incubated for 3 hours being higher than the final nitrogen concentration of the samples that did not have any incubation time (Table 4) and higher than the theoretical N expected in the sample based on inputs. In addition, for both samples that did not have any incubation time (S6a and S7a), the resulting sample was too jelly-like to be used as a liquid fertilizer. Therefore, lack of gelatin breakdown also supports the need for incubation.

### Experiment #3

Increasing amounts of Baker's yeast were tested in combination with Brewer's spent yeast and in addition to water, sucrose (5 g), lysine sulfate (6.25 g), glycine (6.25 g) and gelatin (10 g). Lysine sulfate and glycine were used in this experiment as additional sources of nitrogen that are commercially available, water-soluble and widely distributed globally. The use of these materials within this invention is exemplary only and other amino acids and their salts can also be used in lieu. This selection in no way is it meant to limit the nitrogen-containing compounds and nitrogen-containing low molecular compounds that can be used for the purpose of this invention, nor to limit the scope of the present invention.

The samples prepared (see Table 5) were incubated for 24 hours at 25-30° C. After incubation, all samples were lysed at 50° C. for 24 hours. Cell count was performed on the BSY. Based on cell counts and nitrogen concentration from Experiment #1, a Baker's yeast standard was made with 1:5 of Baker's yeast and water. After lysis, the samples were cooled down to room temperature and weighed before being tested for nitrogen concentration. It will be known to those skilled in the art, that some degree of evaporation occurs during incubation and lysis decreasing the overall mass of the final sample.

TABLE 5
Amounts of additives to samples as per Experiment 3. Baker's
yeast standard was prepared by making a solution
of 1:5 Baker's yeast and water.

| | | | | Baker's |
|---|---|---|---|---|
| Sample | | Water | Baker's | yeast std |
| ID | BSY (g) | (mL) | yeast (g) | (g) |
| P1 | 250 | 100 | 0 | 0 |
| P2 | 187.5 | 100 | 0 | 75 |
| P3 | 125 | 100 | 0 | 150 |
| P4 | 62.5 | 100 | 0 | 225 |
| P5 | 0 | 350 | 50 | 0 |

Samples of Brewer's Spent Yeast (BSY) and Baker's Yeast with no supplementation were sent as controls to determine the nitrogen content within the live cultures. An additional sample with no live cultures were sent to ascertain the nitrogen content derived from the additive. All samples (P1 to P5) were liquid after lysis, demonstrating the ability of the live culture to degrade gelatin at all yeast ratios (Table 6). The extent of degradation of the gelatin was estimated to be estimated to be over 90%. After normalization, the nitrogen in the system is comparable across all ratios (Table 6). Normalization was done by subtracting the grams of nitrogen contributed by the yeast sources. It is thus evidenced, that the addition or replacement of Brewer's Spent yeast with an alternative organism capable of degrading proteins and oligopeptides, as well as capable of performing aerobic respiration, is an appropriate and suitable approach for the manufacturing of a nitrogen-enhanced organic liquid fertilizer.

TABLE 6
Final consistency and nitrogen concentrations of samples with varying
ratios of BSY to Baker's yeast standard. Normalized nitrogen
concentration was done by subtracting the grams of
nitrogen contributed by the yeast sources.

| | Ratio of BSY to | | | Normalized |
|---|---|---|---|---|
| | Baker's yeast | Consistency | Experimental | grams of |
| Sample ID | standard | post-lysis | N % | N in sample |
| P1 | 1:0 | Liquid | 1.57 ± 0.005 | 3.37 ± 0.05 |
| P2 | 3:1 | Liquid | 1.63 ± 0.05 | 3.79 ± 0.18 |
| P3 | 1:1 | Liquid | 1.60 ± 0.02 | 3.56 ± 0.04 |
| P4 | 1:3 | Liquid | 1.62 ± 0.00 | 3.72 ± 0.00 |
| P5 | 0:1 | Liquid | 1.69 ± 0.03 | 3.74 0.07 |

Table 7 sets out an additional analysis of the samples found in Table 6. The data found in Table 7 indicates that the phosphorous and potassium contents, metrics important to fertilizers and denoted typically as part of the N-P-K nomenclature that characterizes fertilizers, vary depending on the organism source used. It appears to indicate that Baker's Yeast does not contain much phosphorus on its own, thus the lower values in samples P4 and P5. However, samples P4 and P5 do contain a slightly higher potassium content than samples containing larger amounts of BSY (P1 and P2). This will be beneficial in different instances where low or high phosphorous or potassium is desired for fertilization. Knowledge of this behavior allows a user to custom tailor the organism blend for the expected fertilizer usage all the while maintaining relatively constant nitrogen content.

TABLE 7
Potassium and phosphorus content for samples P1 to P5

| | K content | P content |
|---|---|---|
| Sample ID | (wt. %) | (wt. %) |
| P1 | 0.58 | 0.25 |
| P2 | 0.63 | 0.20 |
| P3 | 0.53 | 0.27 |
| P4 | 0.84 | 0.06 |
| P5 | 0.84 | 0.04 |

### Experiment #4

In this experiment, a different source of yeast in solid and liquid form was tested in combination with Brewer's spent yeast and in addition to water, sucrose (10 g), lysine sulfate (12.5 g), glycine (12.5 g) and gelatin (20 g). Lysine sulfate and glycine were used in this experiment as additional sources of nitrogen that are commercially available, water-soluble and widely distributed globally. The use of these materials are purely representative of a preferred embodiment of the present invention and other amino acids and their salts can also be used in lieu. This selection in no way is it meant to limit the nitrogen-containing compounds and nitrogen-containing low molecular compounds that can be used for the purpose of this invention, nor to limit the scope of the invention.

The samples prepared (see Table 8) were incubated for 24 hours at 25-30° C. The additional solid yeast was added as a suspension in water. After incubation, all samples were lysed at 50° C. for 24 hours. After lysis, the samples were cooled down to room temperature and weighed before being tested for nitrogen concentration (Table 8). To account for evaporation differences, the nitrogen content was normalized by multiplying the % N by the mass of sample after lysis and reported as grams of N in final sample (Table 8).

TABLE 8
Amounts of additives to samples as per Experiment 4.

| | | | Form of | | | Grams of N in |
|---|---|---|---|---|---|---|
| Sample | | Additional | additional | Relative use | Grams of N in | final sample |
| ID | BSY (g) | yeast (g) | yeast | of BSY (%) | final sample | per g BSY |
| L1/S1 | 500 | 0 | — | 100 | 10.29 ± 0.29 | 0.021 |
| L2 | 450 | 50 | Liquid | 90 | 10.30 ± 0.04 | 0.023 |
| L3 | 400 | 100 | Liquid | 80 | 9.99 ± 0.31 | 0.025 |
| L4 | 300 | 200 | Liquid | 60 | 9.27 ± 0.25 | 0.031 |
| S2 | 450 | 50 | Solid | 90 | 10.31 ± 0.24 | 0.023 |
| S3 | 400 | 100 | Solid | 80 | 10.02 ± 0.09 | 0.025 |
| S4 | 300 | 200 | Solid | 60 | 9.47 ± 0.10 | 0.032 |

Table 8 shows that as the amount of additional yeast, whether as solid or liquid, lowers very slightly the grams of N in the final sample. However, the decrease in N mass in the sample is not significantly impactful considering the impact of lowering the need of BSY. As shown in the column labelled “Grams of N in final sample per g of BSY”, the amount of N obtained per g of BSY increases with the use of the additional yeast, which showcases that the decrease of BSY does not cause a significant impact on the final N content of the fertilizer and that spiking the yeast mixture with a new yeast in addition to the BSY is beneficial in maximizing the amount of fertilizer that can be produced from a given amount of BSY.

### Equivalents and Incorporation by Reference

While the foregoing invention has been described in some detail for purposes of clarity and understanding, it will be appreciated by those skilled in the relevant arts, once they have been made familiar with this disclosure, that various changes in form and detail can be made without departing from the true scope of the invention in the appended claims.

All references, issued patents, and patent applications cited within the body of the present specification are hereby incorporated by reference in their entirety, for all purposes. In particular, Canadian Patent Application No. 3,214,892, filed Sep. 29, 2023 is hereby incorporated by reference in its entirety.

1. A process to make a yeast-based fertilizer composition from a combination of Brewer's Spent yeast material and a second organism, said process comprising the steps of:
a) providing said live Brewer's Spent yeast material;
b) exposing said live Brewer's Spent yeast material to said second organism, wherein said second organism is capable of degrading proteins and performing aerobic respiration, thereby creating a live mixed culture;
c) incubating said live mixed culture in the presence of at least one nitrogen-containing compound thereby creating an incubation mixture;
d) optionally incorporating into said incubation mixture at least one additional nutrient to promote the propagation of said live mixed culture;
e) optionally injecting air into said incubation mixture so as to minimize and/or substantially inhibit the production of ethanol;
wherein said incubation mixture undergoes incubation for a period of time sufficient for said live mixed culture to metabolize said at least one nitrogen-containing compound and for said live mixed culture to propagate and resulting in a pre-lysis mixture comprising metabolized nitrogen-containing compounds and said live mixed culture;
f) autolyzing said pre-lysis mixture under specific conditions yielding an autolyzed mixture which comprises said yeast-based fertilizer composition.

2. The process according to claim 1, further comprising a step of dehydrating or evaporating said autolyzed mixture to yield said yeast-based fertilizer composition with a pre-determined nitrogen-phosphorus-potassium (NPK) number.

3. The process according to claim 1, wherein said yeast-based fertilizer composition is filtered to yield a liquid free of solids having a particle size of 75 microns or greater.

4. The process according to claim 1, wherein said second organism capable of degrading proteins and performing aerobic respiration is a fungus.

5. The process according to claim 4, wherein said fungus belongs to the genus Saccharomyces.

6. The process according to claim 5, wherein said at least one nitrogen-containing compound is selected from: gelatin, tryptone, peptone, collagen, casein, yeast extract, bloodmeal, bonemeal, soybean meal, Lysine (Lys), Methionine (Met), Tryptophan (Trp), Arginine (Arg), Histidine (His), Isoleucine (Ile), Leucine (Leu), Phenylalanine (Phe), Threonine (Thr), Valine (Val), Glycine (Gly), Cysteine (Cys), Tyrosine (Tyr), Alanine (Ala), Glutamine (Gln), Glycine (Gly), Serine (Ser), Asparagine (Asn), Aspartic Acid (Asp), and Glutamic Acid (Glu), and a combination thereof; or is a salt thereof and/or salts thereof.

7. The process according to claim 1 wherein said at least one additional nutrient for the propagation of the live mixed culture is selected from: carbohydrates, vitamins, organic salts, inorganic salts, trace metals, and a combination thereof.

8. The process according to claim 1, wherein the at least one additional nutrient for the propagation of the live mixed culture is a carbohydrate.

9. The process according to claim 1, wherein the step of incubating has a duration ranging from 3 to 48 hours.

10. The process according to claim 1, wherein the step of incubating is carried out at a temperature ranging from 10 to 40° C.

11. The process according to claim 1, wherein the step of autolyzing is carried out at a temperature ranging from 45 to 60° C.

12. The process according to claim 1, wherein a quantity of said nitrogen source added to said live mixed culture in solution ranges from 0.5 to 20% of the total fertilizer composition.

13. The process according to claim 1, wherein the live mixed culture has a dry content ranging from 0 and 30 wt. % of the total fertilizer composition.

14. The process according to claim 1, wherein the process further comprises a step of incorporating at least one low molecular weight nitrogen-containing compound to said pre-lysis mixture-prior to the step of autolyzing.

15. A yeast-based fertilizer composition derived from an incubation of a first organism with a second organism, wherein said first and second organisms are capable of degrading proteins and performing aerobic respiration, and wherein a Brewer's Spent yeast material comprises said first organism and said second organism comes from a different source.

16. The yeast-based fertilizer composition according to claim 15, wherein the incubation of at least said first and said second organisms capable of degrading proteins and perform aerobic respiration, is preceded by an addition of at least one nitrogen-containing compound.

17. The yeast-based fertilizer composition according to claim 15, wherein said second organism is selected from the genus Saccharomyces.

18. The yeast-based fertilizer composition according to claim 15, wherein the incubation of at least two organisms capable of degrading proteins and perform aerobic respiration is preceded by an addition of at least one additional nutrient for the propagation of the live culture selected from: carbohydrates, vitamins, organic salts, inorganic salts, trace metals, and a combination thereof.

19. The yeast-based fertilizer composition according to claim 15, wherein said at least one nitrogen-containing compound is selected from: proteins, amino acids, peptides, amine salts thereof, and a combination thereof.

20. The yeast-based fertilizer composition according to claim 15, wherein said at least one nitrogen-containing compound is selected from: gelatin, tryptone, peptone, collagen, casein, yeast extract, bloodmeal, bonemeal, soybean meal, Lysine (Lys), Methionine (Met), Tryptophan (Trp), Arginine (Arg), Histidine (His), Isoleucine (Ile), Leucine (Leu), Phenylalanine (Phe), Threonine (Thr), Valine (Val), Glycine (Gly), Cysteine (Cys), Tyrosine (Tyr), Alanine (Ala), Glutamine (Gln), Glycine (Gly), Serine (Ser), Asparagine (Asn), Aspartic Acid (Asp), and Glutamic Acid (Glu), and a combination thereof; or is a salt thereof and/or salts thereof.
